(19) United States
(12) Patent Application Publication
(10) Pub. No.: US 2012/0016000 A1
(43) Pub. Date: Jan. 19, 2012

(54) METHOD TO ASSAY PHOSPHOLIPID AND TRIGLYCERIDE TRANSFER ACTIVITY OF MICROSOMAL TRIGLYCERIDE TRANSFER PROTEIN

(76) Inventors: M. Mahmmod Hussain, Woodbury, NY (US); Jahangir Iqbal, Jersey City, NJ (US)

(21) Appl. No.: 13/183,835

(22) Filed: Jul. 15, 2011

Related U.S. Application Data
(60) Provisional application No. 61/364,993, filed on Jul. 16, 2010.

(51) Int. Cl. A61K 31/404; C07D 209/42; A61P 3/06; C40B 30/00; C40B 40/04

(52) U.S. Cl. 514/419; 506/7; 506/15; 548/492

(57) ABSTRACT

The present invention is directed to a method for identifying an antagonist compound of microsomal triglyceride transfer protein (MTP), wherein the antagonist at least partially inhibits triglyceride transfer activity while not significantly inhibiting phospholipids transfer activity of MTP. In particular, the present invention is directed to assays for phospholipid (PL) and triglyceride (TG) transfer activity having considerably improved sensitivity. In addition, antagonist compounds that modulate the lipid transfer activity of MTP are provided. Kits for measuring the lipid transfer activity of MTP are also provided by the present invention.

### CROSS REFERENCE TO RELATED APPLICATIONS

The present application claims the benefit of priority under 35 U.S.C. §119(e) of U.S. Provisional Application No. 61/364,993, filed Jul. 16, 2010.

### GOVERNMENT RIGHTS

This invention was made with government support under grant no. 5R01DK04690014 awarded by the National Institute of Diabetes and Digestive and Kidney Diseases of the National Institutes of Health. The government has certain rights in the invention.

### FIELD OF THE INVENTION

The present invention relates to a high throughput assay for identifying an antagonist compound of microsomal triglyceride transfer protein (MTP), wherein the antagonist at least partially inhibits triglyceride transfer activity while not significantly inhibiting phospholipids transfer activity of MTP. In addition, the present invention relates to methods for treating hyperlipidemias or steatosis in a subject, the method comprising administering to the subject a therapeutically effective amount of an antagonist of MTP which at least partially inhibits triglyceride transfer activity while not significantly inhibiting phospholipid transfer activity of MTP. Kits for measuring the lipid transfer activity of MTP are provided by the present invention.

### BACKGROUND OF THE INVENTION

High plasma lipids are risk factors for various cardiovascular and metabolic disorders such as atherosclerosis, obesity, diabetes, and metabolic syndrome. Lipids are carried in the plasma by large lipid-protein complexes called lipoproteins. Lipoproteins can be classified into two major classes based on the presence and absence of apolipoprotein B (apoB) in these particles. The major apoB-lipoproteins found in human plasma are low-density lipoproteins (LDL); more commonly referred to as “bad cholesterol” in popular press. MTP is the essential chaperone for the assembly and secretion of apoB-lipoproteins as evidenced by the absence of these lipoproteins in the plasma of abetalipoproteinemia subjects that have mutations in the MTTP gene. Although MTP can physically associate with apoB and membranes, its most coveted activity is its ability to transfer neutral lipids. MTP can transfer several lipids in vitro. MTP has been a favorite target to identify small molecule inhibitors and use them to lower plasma lipids. Indeed several MTP antagonists have been identified that decrease lipoprotein production and plasma lipids. However, inhibition of MTP has been associated with significant side effects,

Kinetics studies have suggested the presence of two binding sites for triglycerides and phospholipids in MTP. Further, evolutionary studies indicate that MTP evolved as a phospholipid transfer protein and acquired triglyceride transfer activity during a transition from invertebrate to vertebrate. Based on these kinetic and evolutionary studies, it is hypothesized that MTP has two different lipid transfer sites: a high affinity-binding site for triglycerides and phospholipids and a second low affinity binding site for phospholipids only. Compounds that inhibit triglyceride transfer and spare phospholipid transfer activity might be ideal to lower plasma lipids, to reduce obesity, and to regress atherosclerosis because they may lack side effects associated with antagonists that inhibit both the lipid transfer activities of MTP.

What is needed is the identification of antagonists that inhibit the high affinity site but do not affect the low affinity site. Further, what is needed is the identification of MTP inhibitors with none to moderate side effects that would be ideal for the treatment of homozygous familial hypercholesterolemia and overtly obese patients. Inhibition of MTP is expected to decrease lipoprotein assembly and secretion thereby reducing plasma lipid levels. Use of specific antagonists that inhibit triglyceride transfer activity of MTP without affecting its phospholipid transfer activity may avoid toxicities associated with accumulation of lipids in different tissues. In addition, identification of such compounds would provide evidence for the existence of two functionally independent domains involved in phospholipid and triglyceride transfer in MTP.

The present invention is directed to a high throughput screening (HTP) assay with enhanced sensitivity, ease of use, rapidity, selectivity, versatility and avoidance of the use of negatively charged lipids that inhibit MTP activity for identifying an antagonist compound of MTP wherein the antagonist at least partially inhibits triglyceride transfer activity while not significantly inhibiting phospholipids transfer activity of MTP. The present invention is also directed to a method by using an antagonist compound for lowering the high plasma lipids and lipoproteins in the blood of a patient without the negative of effects associated with antagonists that inhibit both the lipid transfer activities of MTP.

### SUMMARY OF THE INVENTION

The present invention is directed to a method for identifying an antagonist of MTP, wherein the antagonist at least partially inhibits triglyceride transfer activity while not significantly inhibiting phospholipids transfer activity of MTP. In particular, the method of the present invention comprises the following steps:

(a) identifying said antagonist in a primary high throughput (HTP) assay by:
(i) adding MTP into a cocktail comprising donor and acceptor lipid vesicles;
(ii) eliminating compounds which fluoresce and interfere with fluorescence detection by quenching or enhancing fluorescence;
(iii) identifying remaining compounds after step (ii) which at least partially inhibit triglyceride transfer activity of MTP; and
(iv) identifying remaining compounds after step (iii) which at least partially inhibit phospholipid transfer activity of MTP, resulting in identification of said antagonist;
   (b) validating the antagonists identified in step (a)(iv) in a secondary radiolabeled lipid transfer assay by:
(i) preparing donor lipid vesicles comprising radiolabeled lipids and negatively charged cardiolipin;
(ii) preparing acceptor lipid vesicles;
(iii) incubating MTP with the donor lipid vesicles and acceptor lipid vesicles;
(iv) removing the donor lipid vesicles and MTP; and
(v) measuring net transfer of radiolabeled lipids by MTP from the donor to acceptor lipid vesicles by measuring radioactivity in the acceptor vesicles; and
   (c) characterizing the biological activity of the identified antagonist obtained from the primary assay of step (a)(iv).

An additional validation assays can be conducted to determine the biological activity of the compounds identified in steps (a) and (b). This type of assay can be used to determine the biological activity of the compounds obtained during the primary and secondary assays, (steps (a) and (b)), discussed above and are cell-based assays. MTP and apoB expressing cell lines (human hepatoma HepG2 and Huh7 as well as colon carcinoma Caco-2 cells) will be incubated with these compounds and secretion of apoB into the media will be studied at different time points. At the end of the experiment, these cells will be used to measure both triglyceride and phospholipid transfer activities of MTP. To further validate the utility of these compounds in lowering plasma lipids, they will be fed to mice and reductions in plasma lipid levels will be documented.

Identification of compounds that inhibit triglyceride transfer activity with no to moderate effect on phospholipid transfer activity of MTP would provide evidence for the existence of two different functional domains in MTP that transfer two different types of lipids. Compounds that inhibit triglyceride transfer and spare phospholipid transfer activity might be ideal to lower plasma lipids, to reduce obesity, and to regress atherosclerosis because they may lack side effects associated with antagonists that inhibit both the lipid transfer activities of MTP.

The present invention is also directed to a method for treating hyperlipidemias or steatosis in a subject. In particular, the method of the present invention comprises administering to the subject a therapeutically effective amount of an antagonist of MTP which at least partially inhibits triglyceride transfer activity of MTP while not significantly inhibiting phospholipid transfer activity of MTP.

The present invention is also directed to an antagonist that at least partially inhibits triglyceride transfer activity of MTP while not significantly inhibiting phospholipid transfer activity of microsomal MTP.

Variations and additions to the methods described above are also part of the present invention and are described in greater detail in the Detailed Description of the Invention section including the examples and figures below.

### BRIEF DESCRIPTION OF THE FIGURES

FIG. 1 provides a figure of a molecule that would inhibit triglyceride transfer activity (i.e., lipid transfer domain) while retaining significant phospholipid transfer activity (i.e., membrane binding domain) of MTP.

FIGS. 2A, 2B, 2C, 2D and 2E provide graphical representations of the effects of time, concentrations of MTP, and temperature for triglyceride (NBD-TG) and phospholipid (NBD-PE) transfer activities of purified bovine liver MTP.

FIGS. 3A and 3B provide graphical representations of the effect of increasing amounts of solvent dimethyl sulfoxide (DMSO) on the assay tolerance on triglyceride (NBD-TG) and phospholipid (i.e., NBD-PE) transfer activities of MTP.

FIGS. 4A and 4B provide graphical representations of the evaluation of signal-to-noise ratio (′Z) for triglyceride (NBD-TG) and phospholipid (NBD-PE) transfer activities of MTP.

FIGS. 5A and 5B provide graphical representations of the dose-dependent effect of known MTP antagonist (CP-346086) on inhibition assays of triglyceride (NBD-TG) and phospholipid (NBD-PE) transfer activities of MTP.

FIGS. 6A, 6B, 6C and 6D provide graphical representations of the variations between day-to-day (FIGS. 6A and 6B) and plate (FIGS. 6C and 6D) differences in the triglyceride (NBD-TG) and phospholipid (NBD-PE) transfer activity assays of MTP.

### DETAILED DESCRIPTION OF THE INVENTION

Microsomal triglyceride transfer protein (MTP) is a possible therapeutic target in treating hyperlipidemias, but its inhibition is associated with side effects. Identification of MTP inhibitors with none to moderate side effects is highly desirable which would be ideal for the treatment of homozygous familial hypercholesterolemia and overtly obese patients. These compounds can possibly be used in a larger hyperlipidemic population.

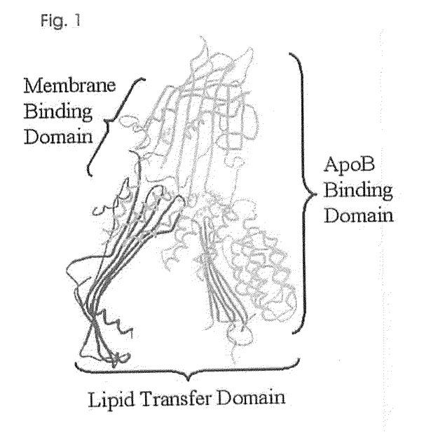

Envisioned are several additional uses for the compounds identified in the proposed HTS for MTP. The compounds can be used for follow-up research programs both in biological research and therapeutic development. In biological research, the compounds can be used to modulate the lipid transfer activity in vitro and in vivo, and to study the mechanisms involved in lipid binding, lipid transfer and net deposition by MTP. Furthermore, the compounds can be evaluated to differentiate between different functional activities, such as lipid transfer, apoB binding, and vesicle association. These compounds could be unique sources in identifying different pockets in the MTP molecule (FIG. 1) involved in the transfer of phospholipids and neutral lipids. For therapeutic use, these compounds may serve as lead compounds and may provide a prototype for the development of more potent antagonists.

Inhibition of MTP is expected to decrease lipoprotein assembly and secretion thereby reducing plasma lipid levels. Use of specific antagonists that inhibit triglyceride transfer activity of MTP without affecting its phospholipid transfer activity may avoid toxicities associated with accumulation of lipids in different tissues. In addition, identification of such compounds would provide evidence for the existence of two functionally independent domains involved in phospholipid and triglyceride transfer in MTP.

### Microsomal Triglyceride Transfer Protein (MTP):

Abetalipoproteinemia is characterized by the absence of plasma apoB-lipoproteins, extremely low plasma cholesterol, and lipid soluble vitamin deficiencies. Using various genetic approaches it has been showed that afflicted individuals have mutations in the MTTP gene. Several mutations in the MTTP gene have since been documented in abetalipoproteinemia. Tissue specific liver knockout models recreated the apoB and lipid deficiencies present in abetalipoproteinemia. Furthermore, cell culture studies showed that wild type MTP can rescue apoB secretion but mutated proteins cannot. These studies indicate that MTP is essential for intracellular lipoprotein assembly. MTP is required during the early stages of assembly to prevent the aberrant folding of apoB and its degradation by proteasomes.

MTP is a heterodimer of 97 kDa MTP and 55 kDa PDI subunits. Based on sequence homology with lipovitellin, the MTP subunit is proposed to contain three structural domains (FIG. 1): N-terminal β-barrel, central α-helical domain, and C-terminal lipid-transfer cavity, which might carry out three independent functions. Antagonists that differentially inhibit the lipid transfer and apoB-binding activities have been identified. There is evidence to indicate that the “lipid transfer domain” might be the major site involved in lipid transfer. Our hypothesis is that the “membrane-binding domain” may act as an additional site to transfer phospholipids. In fact, lipovitellin crystals have few phospholipids in this domain. The present invention is directed to a method to identify molecules that would inhibit triglyceride transfer activity (FIG. 1, Lipid transfer domain) while retaining significant phospholipid transfer activity (FIG. 1, Membrane binding domain) of MTP, which activity is sufficient to support apoB-lipoprotein assembly and secretion.

### Different Sites in MTP May be Involved in the Transfer of Triglycerides and Phospholipids:

MTP can transfer several lipids in vitro. Kinetics studies have shown that MTP is a lipid transfer protein and transfers lipids by ping-pong bi-bi shuttle mechanism. According to this mechanism, MTP interacts with donor membranes, extracts lipids, interacts with acceptor membranes, and deposits lipids in acceptor membranes. Further studies revealed that transfer of triglyceride was fast and complete, whereas phospholipid transfer showed biphasic transfer kinetics consisting of fast and slow phases. It has been proposed that MTP has two different lipid transfer sites: a high affinity-binding site for triglycerides and phospholipids and a second low affinity binding site for phospholipids only. The present invention is also directed to the identification of antagonists that inhibit the high affinity site but do not affect the low affinity site.

Evidence for the possible existence of two different binding sites also comes from studies about the evolution of MTP. It has been found that Drosophila MTP transfers phospholipids but does not transfer triglycerides. Despite the lack of triglyceride transfer activity, Drosophila MTP assists in the assembly and secretion of apoB-lipoproteins. Therefore, MTP evolved as a phospholipid transfer protein and this activity is sufficient to assist in apoB-lipoprotein assembly. Further studies in several species revealed that zebra fish have some ability to transfer triglycerides. Moderate and maximum triglyceride transfer activities were seen in the livers of frogs and birds, respectively. Specific triglyceride transfer activities in the livers of rodents and monkeys were similar to those seen in birds. Therefore, it appears that MTP evolved as a phospholipid transfer protein and then acquired triglyceride transfer activity during a transition from invertebrate to vertebrate. This activity was optimized in birds and then retained in mammals.

These conclusions demonstrate that there was one phospholipid site and another site was added on to transfer triglyceride. It is also possible that the same phospholipid transfer site was evolved to accommodate triglycerides. Based on kinetics data, it could also be indicated that both the retention of ancient phospholipid transfer activity and acquisition of a new triglyceride transfer site might have occurred. Distinguishing these different sites based on various bioinformatics and molecular approaches has not been possible. Identification of inhibitors that specifically inhibit either of these activities would provide evidence that MTP contains two functionally independent domains involved in phospholipid and triglyceride transfer activities.

### MTP, a Target to Treat Hyperlipidemias:

Hyperlipidemias are major risk factors for atherosclerosis. There are two metabolic abnormalities that could lead to hyperlipidemias, over production or decreased catabolism. Significant progress has been made with statins in lowering plasma lipids by increasing their catabolism. However, attempts to control lipoprotein production have not yet been successful. ApoB and MTP are prime candidates to curb lipoprotein production. Since apoB does not have a biochemical activity amenable to HTS, siRNA technology has been used to lower its production.

In contrast to apoB, due to its lipid transfer activity, MTP has been a favorite target to identify small molecule inhibitors and use them to lower plasma lipids. Indeed several MTP antagonists have been identified that decrease lipoprotein production and plasma lipids. Inhibition of MTP has been associated with significant side effects. Therefore, none of the MTP inhibitors have been approved for therapeutic use. Nevertheless, they have been evaluated for use in familial hypercholesterolemia and in moderate hypercholesterolemic patients. It is anticipated that MTP inhibitors might also be useful in treating overt obesity. The use of these drugs in homozygous familial hypercholesterolemia and possibly in the treatment of overt obesity is justifiable on the grounds that the alternate choices for their treatments are liver or gastric bypass surgeries. It should be pointed out that MTP inhibitor, dirlotapide, is currently used in canines to decrease lipid absorption and reduce weight.

MTP inhibitors exhibit two types of side effects related to accumulation of fat (steatosis) in cells that produce apoB-lipoproteins. The first side effect is related to the inhibition of lipoprotein assembly by enterocytes and manifests as gastrointestinal disturbances such as steatorrhea and diarrhea. These disturbances have been avoided by administering MTP inhibitors 4 h after the supper. Recently, IRE1β has been shown to down regulate intestinal MTP indicating that its up-regulation might be a viable target for lowering intestinal MTP. The second side effect is related to the inhibition of hepatic lipoprotein assembly and secretion. In about 10-30% of the individuals, MTP inhibitors increase plasma levels of liver enzymes, mainly aspartate (AST) and alanine (AST) aminotransferase. Thus, there is a critical need for novel approaches to inhibit MTP without causing steatosis. It is proposed that intestine-specific inhibition of MTP might be beneficial because of the inherent property of the intestine to self-renew. In fact, intestine-specific MTP inhibitor, JTT-130, has been shown to lower plasma triglyceride and LDL cholesterol in guinea pigs without increasing hepatic triglyceride. Similarly, another intestine-specific compound, SLx-4090, has been shown to lower plasma lipids.

Significant progress is being made to understand molecular mechanisms behind toxicities associated with chemical inhibition or gene ablation of MTP. A major clue about the reasons for the toxicities associated with MTP inhibition came from the observations that MTP, besides being essential for apoB-lipoprotein biosynthesis, also plays an important role in cholesteryl ester biosynthesis. Inhibition of MTP leads to significant accumulation of free cholesterol in the livers. Therefore, toxicities associated with MTP inhibition or ablation might be related with the accumulation of free cholesterol. It has also been found that hepatotoxic effects of MTP ablation can also be avoided by expressing Drosophila MTP in liver-specific MTP knockout mice (Khatun et al, manuscript in preparation). Mice deficient in MTP accumulate significant amounts of triglyceride and free cholesterol. We had hypothesized that expression of DrosophilaMTP would result in the biosynthesis of apoB-lipoproteins. These lipoproteins would be phospholipid-rich. We had also anticipated that hepatic triglyceride will not be affected by the expression of Drosophila MTP because it does not transfer triglyceride. As expected, expression of Drosophila MTP resulted in the biosynthesis of apoB-lipoproteins by the liver. Unexpectedly, it also significantly reduced liver triglycerides. The lipoproteins synthesized in the presence of Drosophila MTP were phospholipid-rich, but they did carry some triglyceride. It is likely that some triglycerides associate with nascent apoB-lipoproteins during desorption of nascent lipoproteins from the membranes (primary site of lipoprotein assembly) of endoplasmic reticulum. These unexpected findings indicate that phospholipid transfer activity might be sufficient for normal biological processing of triglycerides and that triglyceride transfer activity of MTP is dispensable. Therefore, identification of small chemical compounds that inhibit triglyceride transfer activity of MTP without affecting its phospholipid transfer activity are considered ideal to reduce hyperlipidemia (high plasma lipids) and steatosis (high tissue lipids).

MTP Assays and their Utility in HTS:

The present invention is directed to very selective, simple, rapid and sensitive fluorescence assays to measure triglyceride (NBD-TG) and phospholipid (NBD-PE) transfer by MTP. These assays have been modified and developed into a “mix and measure” format kit that is stable, user friendly, and consists of only two components: a master mix and purified MTP. The master mix contains all the ingredients necessary for MTP activity. In this assay, MTP transfers fluorescent lipids from donor to acceptor vesicles. Lipid fluorescence in these vesicles is mostly quenched. Unquenched fluorescence (˜0.05% of total fluorescence) is measured as background. During the transfer of fluorescent lipids by MTP, fluorescence is exposed to aqueous phase and detected by the Fluorimeter. These fluorescence measurements represent the amount being transferred. Inclusion of antagonist during these assays reduces increases in fluorescence and represents inhibition of the transfer activity. Optimization of the chemical composition of both donor and vesicles significantly enhanced the sensitivity of the assays. The data presented in preliminary studies below demonstrate that the assays are highly suitable for HTS.

The present invention is directed to identification of compounds that inhibit triglyceride activity of MTP and spare its phospholipid transfer activity using HTS:

Primary HTS to Identify Chemical Probes that Inhibit the Triglyceride, but not the Phospholipid, Transfer Activity of MTP:

The primary HTS assays of the present invention are based on time-dependent increases in fluorescence after the addition of MTP into a cocktail consisting of donor and acceptor vesicles. The assays were performed in 100 μl volume in a 96-well format. These assays can be easily adopted for HTS of small molecule libraries against MTP in 384- or 1536-well plates. Identification of compounds that inhibit triglyceride transfer activity without affecting its ability to transfer phospholipids using HTS by the following steps. First, compounds that fluoresce and interfere with fluorescence detection will be eliminated. Second, compounds that inhibit >50% of the triglyceride transfer activity will be identified. Third, these compounds will be evaluated for their inability or less potency to inhibit phospholipid transfer activity. Chemical probes that inhibit >50% of triglyceride transfer activity and <25% of the phospholipid transfer activity will be selected for further evaluations.

### Validation of Hits Generated by Primary HTS Assays Using Alternate Radiolabeled Lipid Transfer Assays:

To validate positive hits by an alternate assay, radiolabeled lipid transfer assays are used that are based on the net transfer of radiolabeled lipids from the donor to acceptor vesicles. Donor vesicles in these assays will contain radiolabeled lipids. In addition, the donor vesicles will also have negatively charged cardiolipin to facilitate their separation from acceptor vesicles at the end of the reaction. At the end of the reaction, donor vesicles are separated using DE-52. The net transfer of lipids by MTP to acceptor vesicles is then quantified by measuring the radioactivity in the acceptor vesicles. It will be demonstrated that these compounds do not inhibit other cellular and plasma lipid transfer proteins.

### Characterization of the Biological Activity of the Identified Chemical Probes:

To determine the biological activity of the compounds obtained during the primary and secondary assays, cell-based assays are used. MTP and apoB expressing cell lines (human hepatoma HepG2 and Huh7 as well as colon carcinoma Caco-2 cells) are incubated with these compounds and secretion of apoB into the media is studied at different time points. At the end of the experiment, these cells are used to measure both triglyceride and phospholipid transfer activities of MTP. To further validate the utility of these compounds in lowering plasma lipids, they are fed to mice and reductions in plasma lipid levels are documented.

In one embodiment of the present invention, the method for identifying an antagonist of microsomal triglyceride transfer protein (MTP), wherein the antagonist at least partially inhibits triglyceride transfer activity while not significantly inhibiting phospholipid transfer activity of MTP, is described below using the following steps:

(a) identifying said antagonist in a primary high throughput (HTP) assay by:
(i) adding MTP into a cocktail comprising donor and acceptor lipid vesicles;
(ii) eliminating compounds which fluoresce and interfere with fluorescence detection by quenching or enhancing fluorescence;
(iii) identifying remaining compounds after step (ii) which at least partially inhibit triglyceride transfer activity of MTP; and
(iv) identifying remaining compounds after step (iii) which partially inhibits phospholipid transfer activity of MTP, resulting in identification of said antagonist;
   (b) validating the antagonists identified in step (a)(iv) in a secondary radiolabeled lipid transfer assay by:
(i) preparing donor lipid vesicles comprising radiolabeled lipids and negatively charged cardiolipin;
(ii) preparing acceptor lipid vesicles;
(iii) incubating MTP with the donor lipid vesicles and acceptor lipid vesicles;
(iv) removing the donor lipid vesicles and MTP; and
(v) measuring net transfer of radiolabeled lipids by MTP from the donor to acceptor lipid vesicles by measuring radioactivity in the acceptor vesicles; and
   (c) characterizing the biological activity of the identified antagonist obtained from the primary assay of step (a)(iv).

According to an aspect of the above embodiment, the compounds identified in step (a)(iii) inhibit greater than 50%, greater than 60%, greater than 70%, preferably greater than 80%, preferably greater than 90% or most preferably 100% of triglyceride transfer activity of MTP, while the compounds identified in step (a)(iv) inhibit less than 25%, less than 20%, less than 15%, preferably less than 10%, preferably less than 5% or most preferably 0% s of phospholipid transfer activity of MTP.

According to an aspect of the above embodiment, the compounds identified in step (a)(iii) inhibit in the ranges of 50% to 100%, 60% to 100%, 70% to 100%, preferably 80% to 100%, preferably 90% to 100%, or most preferably 100% of phospholipid transfer activity of MTP, while the compounds identified in step (a)(iv) inhibit in the ranges of 0% to 25%, 0% to 20%, 0% to 15%, preferably 0% to 10%, preferably 0% to 5% or most preferably 0% of phospholipid transfer activity of MTP.

In another embodiment of the present invention, the method for treating hyperlipidemias or steatosis in a subject comprises administering to the subject a therapeutically effective amount of an antagonist of MTP which at least partially inhibits triglyceride transfer activity while not significantly inhibiting phospholipid transfer activity of MTP.

According to an aspect of the above embodiment, the antagonist inhibits greater than 50%, greater than 60%, greater than 70%, preferably greater than 80%, preferably greater than 90% or most preferably 100% of triglyceride transfer activity of MTP, while the antagonist inhibits phospholipid transfer activity of MTP less than 25%, less than 20%, less than 15%, preferably less than 10%, preferably less than 5% or most preferably 0%.

According to an aspect of the above embodiment, the antagonist inhibits triglyceride transfer activity of MTP in the ranges of 50% to 100%, 60% to 100%, 70% to 100%, preferably 80% to 100%, preferably 90% to 100%, or most preferably 100%, while the antagonist inhibits phospholipid transfer activity of MTP in the ranges of 0% to 25%, 0% to 20%, 0% to 15%, preferably 0% to 10%, preferably 0% to 5% or most preferably 0%.

In another embodiment of the present invention, the antagonist compound of MTP identified by the assays of the present invention at least partially inhibit triglyceride transfer activity while not significantly inhibiting phospholipid transfer activity of microsomal MTP.

According to an aspect of the above embodiment, the antagonist inhibits greater than 50%, greater than 60%, greater than 70%, preferably greater than 80%, preferably greater than 90% or most preferably 100% of triglyceride transfer activity of MTP, while the antagonist inhibits phospholipid transfer activity of MTP less than 25%, less than 20%, less than 15%, preferably less than 10%, preferably less than 5% or most preferably 0%.

According to an aspect of the above embodiment, the antagonist inhibits triglyceride transfer activity of MTP in the ranges of 50% to 100%, 60% to 100%, 70% to 100%, preferably 80% to 100%, preferably 90% to 100%, or most preferably 100%, while the antagonist inhibits phospholipid transfer activity of MTP in the ranges of 0% to 25%, 0% to 20%, 0% to 15%, preferably 0% to 10%, preferably 0% to 5% or most preferably 0%.

In another embodiment of the present invention, a kit for measuring triglyceride and phospholipid transfer activity of MTP is provided comprising a master mix and purified MTP, wherein the master mix comprises acceptor vesicles and fluorescence-labeled donor vesicles.

### RESEARCH DESIGN AND METHODS

Primary HTS to Identify Molecules that Inhibit Triglyceride, but not Phospholipids, Transfer Activity of MTP:
Described below is a multi-step strategy to identify molecules that inhibit triglyceride but not phospholipids or transfer activity of MTP.
i. Elimination of fluorescent compounds: Since the assay is based on fluorescence detection, compounds with inherent fluorescent properties will interfere with the assay. Therefore, fluorescence of the compounds is first measured using 465 nm excitation and 535 nm emission wavelengths. All the compounds that have measurable readings at these wavelengths will not be used for further screening. If required, these eliminated compounds are screened using radiolabel assays for additional evaluations.
ii. Elimination of compounds that interfere with fluorescence detection: A concern of the HTS is the identification of “false positives”. In the assay, false positives may originate because the compounds quench fluorescence instead of inhibiting the MTP activity. Therefore, the remaining non-fluorescent compounds are checked for their ability to quench NBD fluorescence. During these assays, information is gathered about the compounds that enhance fluorescence. Compounds that quench or enhance fluorescence are not used for screening against MTP. As stated before, if need arises, these eliminated compounds are evaluated using a radiolabel assay. After these two preliminary screenings using intact (to measure effects on background fluorescence) and disrupted (to measure effects on total fluorescence) donor and acceptor vesicles, the remaining compounds are screened for their ability to inhibit MTP activity.
iii. Screening of compounds for the inhibition of triglyceride transfer activity: Compounds that inhibit NBD-triglyceride transfer activity of MTP are first identified. For this purpose, different compounds [10 μM] are added during MTP triglyceride transfer assay. The final concentration chosen depends upon the actual assay performance determined during assay implementation at MPLSCN, which is capable of achieving 5-20 μM without intermediate dilutions. Readings are collected after 30 min incubations at room temperature. Parallel incubations include vesicles without MTP (Background) and vesicles with isopropanol (Total fluorescence). Compounds that inhibit >50% of the triglyceride transfer activity are subjected to additional screening. During this second round of screening, selected compounds are evaluated in duplicate for their ability to inhibit triglyceride transfer activity of MTP. Compounds that consistently inhibit MTP activity will be selected for further evaluations.

It is anticipated that compounds that inhibit triglyceride transfer activity with interact with the high affinity lipid transfer domain (FIG. 1). Therefore, some of the phospholipid transfer activity is inhibited. In fact, most of the compounds identified by pharmaceutical industry inhibit both the lipid transfer activities to similar extent. It is speculated that this was mainly due to optimization of chemical probes for maximum inhibition of lipid transfer activities. Therefore, the approach of the present invention is novel in that compounds that inhibit triglyceride transfer activity but do not inhibit or partially inhibit phospholipid transfer activity are identified. The partial inhibition of triglyceride transfer activity is thought to be beneficial in order to keep some residual lipoprotein biosynthetic capacity intact in the liver and intestine.

iv. Screening of selected compounds for their ability to inhibit phospholipid transfer activity of MTP: Experiments are performed to determine whether the identified compounds inhibit transfer of NBD-PE by MTP. For this purpose, a constant concentration of different compounds (10 μM) is added to a reaction mixture that determines phospholipid transfer activity of MTP. Readings are collected after 180 min incubations at 37° C. Parallel incubations will include vesicles without MTP (Background) and vesicles with isopropanol (Total fluorescence). Compounds that inhibit >25% of the phospholipid transfer activity are discarded. Compounds that inhibit less than 25% of the phospholipid transfer activity are used for additional screening. During this additional screening, selected compounds are evaluated in duplicate for their consistent ability to inhibit phospholipid transfer activity transfer activity of MTP.

It is preferable to obtain compounds that have no inhibitory activity against the phospholipid transfer activity of MTP. However, compounds that only partially (<25%) inhibit phospholipid transfer activity are acceptable. After selecting compounds for these properties, full dose response inhibition studies are performed over a 3 log concentrations using 11 different concentrations. Ideally, compounds that would have IC₅₀ values for triglyceride transfer activity that are 5-10 fold higher than those for its phospholipid transfer activity are selected.

Selected chemical probes are evaluated for their efficacy in inhibiting MTP activity using cell lysates obtained from human hepatoma cells. From these studies, compounds that inhibit >50% of triglyceride transfer activity and <25% of phospholipid transfer activity of purified bovine MTP as wels as partially purified human MTP are identified. Next, these properties are validated using a completely independent approach.

### Validation of Hits Generated by Primary HTS Assays Using Alternate Radiolabeled Lipid Transfer Assays:

To validate any positive hits and to rule out false positives generated during the HTS, alternate assays are used that measure net transfer of radiolabeled lipids by MTP from the donor to acceptor vesicles. Donor vesicles are prepared containing either radiolabeled triolein or phosphatidylethanolamine. Donor vesicles will also contain negatively charged cardiolipin that are used to separate the donor vesicles from the acceptor vesicles after the transfer of lipids by the MTP. After incubation of MTP with inhibitors and vesicles for 4 h, donor vesicles and MTP are removed by the addition of DE-52. After centrifugation, radioactivity in the supernatants are quantified. The net transfer of radiolabeled lipids to acceptor vesicles (% of radiolabel transferred) are calculated. Counts obtained in the presence of antagonists are used to calculate % inhibition. Again, compounds that inhibit triglyceride transfer by >50% and phospholipid transfer by <25% at 10 μM concentration are used for further analyses.

To establish the specificity of the identified compounds towards MTP, counter screening is performed to ensure that these compounds do not inhibit other known plasma and cellular lipid transfer proteins. For this purpose, the effect of identified compounds against cholesterol ester transfer protein, phospholipid transfer protein, and phosphatidylinositol transfer protein is evaluated. After this counter screening, compounds are further checked for undesirable biopharmaceutical properties and those that inhibit P450 enzymes, e.g. CYP 3A4, CYP 2D6, CYP2C9, are discarded. After the completion of these studies, the biological activity of the identified chemical probes or inhibitors is checked.

### Characterization of the Biological Efficacy of the Identified Chemical Probes:

To determine the specificity of the compounds obtained during the above HTS, cell-based assays are used. In these assays MTP and apoB expressing cell lines (human hepatoma cell lines, HepG2 and Huh7, and colon carcinoma Caco-2 cells) are incubated overnight in triplicate with these compounds at 0-100 μM concentrations and secretion of apoB into the media is studied. At the end of the experiment, cells are used to measure both triglyceride and phospholipid transfer activities of MTP. Moreover, lipids present in cells to examine whether these compounds cause steatosis are quantified. To control for cellular toxicity, secretion of apoAl and release of AST, ALT, and LDH is measured. Compounds that do not show any cellular toxicity but significantly (>50%) decrease apoB secretion are then used for preclinical studies.

For preclinical studies, mice fed a western diet for 2 months are used. These mice have high plasma triglyceride and cholesterol compared to those fed a chow diet. Different concentrations (0-100 mg/kg) of these compounds will be fed daily for 1 week to these high fat diet fed mice (n=5) and plasma lipid levels will be measured on day 8 after an overnight fast. Plasma will be used to measure AST, ALT, and LDH. Liver, intestine, heart, and kidneys will be collected to measure cellular lipids. The data obtained in these studies will be used to determine whether the identified compounds decrease plasma lipids and avoid steatosis.

### Preliminary Studies and Results

### Time and Temperature Dependence of the MTP Assay:

Black 96-well plates are used to measure the triglyceride and phospholipid transfer activities of purified MTP. Donor vesicles used for triglyceride transfer assay were comprised of 966 pmoles of phosphatidylcholine and 150 pmoles of NBD-TG. For phospholipid transfer assay, donor vesicles contained 161 pmoles of phosphatidylcholine, 204 pmoles of phosphatidylethanolamine, 83 pmoles of triolein, and 171 pmoles of NBD-PE. Acceptor vesicles used in both of these assays comprised of 5260 pmoles of phosphatidylcholine and 1120 pmoles of phosphatidylethanolamine.

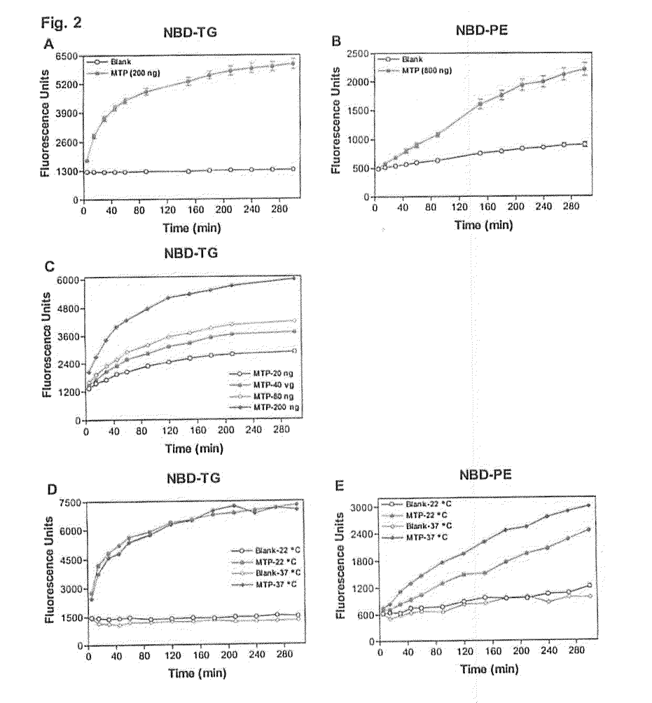

For triglyceride and phospholipid transfer activities, 200 ng and 800 ng, respectively, were used of purified bovine liver MTP. The effects of time, concentrations of MTP, and temperature on MTP activity are shown in FIG. 2. Both triglyceride (FIG. 2A) and phospholipid (FIG. 2B) transfer activities of MTP increased with time. The triglyceride transfer activity progress curve of MTP looked biphasic consisting of fast (t1/2, ˜10 min) and slow (t1/2, ˜120 min) rates (FIG. 2A). On the other hand, phospholipid transfer activity of MTP was linear at all time points (FIG. 2B).

To probe further the kinetics of triglyceride transfer, different amounts of MTP were used. At all concentrations used, the triglyceride transfer activity showed biphasic curve (FIG. 2C) indicating that this might be an inherent property of MTP. This probably represents the fast transfer of triglycerides by MTP. Since data with 200 ng of MTP gave significant transfer until 30 min, these conditions were used in all the subsequent experiments.

Next, the effect of temperature on these transfer activities were studied. Triglyceride transfer activity of MTP (FIG. 2D) was the same at room temperature (22° C.) and at 37° C., indicating no significant effect of temperature on the activity. However, phospholipid transfer activity of MTP (FIG. 2E) was more at 37° C. compared to 22° C. but was linear at both the temperatures tested. These results indicate that these lipid transfer activities exhibit different kinetic properties.

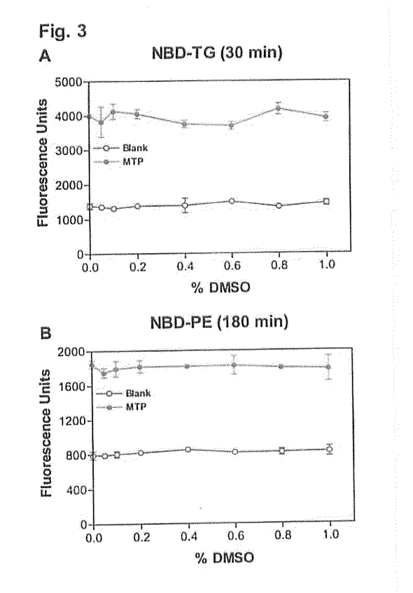

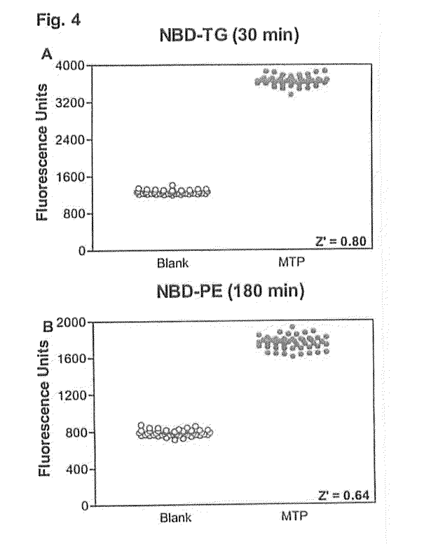

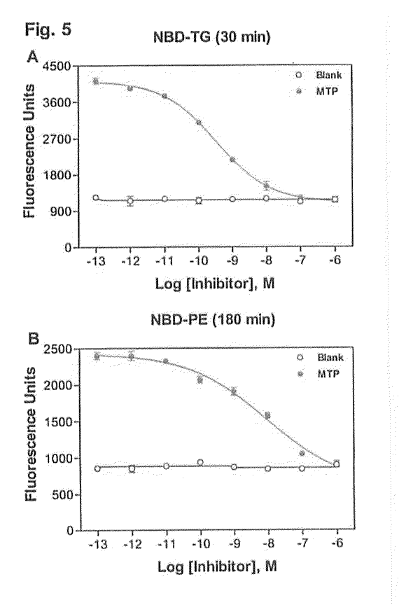

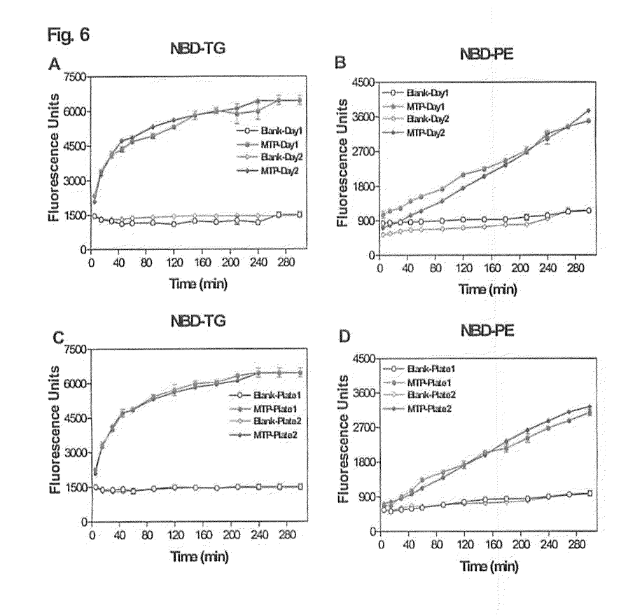

Effect of DMSO on the assay tolerance: The effect of increasing amounts of DMSO on MTP activity is shown in FIG. 3. Increasing amounts of DMSO (0-1%) had no significant effect on both triglyceride (FIG. 3A) and phospholipid (FIG. 3B) transfer activities of MTP. These results indicate that both the assays can tolerate 1%, and possibly higher concentrations, of DMSO.
Evaluation of signal-to-noise ratio of assay across the entire 96-well plate: Fluorescence in the blank (n=48) and MTP (n=48) was used to determine the values of noise and signal, respectively. Determination of Z′ factor for triglyceride (FIG. 4A, Z′=0.80) and phospholipid (FIG. 4B, Z′=0.64) transfer activities of MTP demonstrated that these assays are highly suitable for the HTS.
Dose-dependent effect of known MTP antagonist on the assays: To determine the specificity of the assays, dose-dependent inhibition assays were performed using a known MTP antagonist (CP-346086). With increasing concentration of the antagonist, there was a dose-dependent inhibition of both triglyceride (FIG. 5A) and phospholipid (FIG. 5B) transfer activities of MTP. The IC₅₀ values for the inhibition of triglyceride and phospholipid transfer activities were 0.33 nM and 9.4 nM, respectively. These data indicate that these assays respond to known antagonists of MTP.
Between plate and day-to-day assay variations: Between plates variation were obtained by performing these assays in two different plates at the same time and day-to-day variations were obtained by performing these assays on two alternate days. There were no significant day-to-day (FIGS. 6A and 6B) or between plate (FIGS. 6C and 6D) differences in the triglyceride (FIGS. 6A and 6C) and phospholipid transfer (FIGS. 6B and 6D) activity assays of MTP.
Description of hit validation assay: The primary HTS assays are based on the transfer of NBD-labeled lipids from the donor to the acceptor vesicles. The assay requires pipetting of three solutions: donor and acceptor vesicle mix, the MTP source and water to make up the volume. In drug screening, an additional pipetting step for different compounds is required. For HTS assay three different conditions (blank, positive control, and chemical probe) are recommended. In all assays, the reaction is started by the final addition of the MTP source. The preliminary data was collected in a reaction volume of 100 μl using a 96-well plate format. Donor and acceptor vesicle mix (5 μl) was pipetted into a 96-well fluorescence microtiter (black) plates. In blanks, a needed amount of control buffer (that contains the MTP source in a positive control) and solvent e.g., DMSO etc. (that contains the chemical probe) was added and the volume was made up with water to 100 μl. In the positive control, a known amount of the MTP source and a needed amount of solvent was added and the volume was made up to 100 μl. In chemical probe, a known amount of the MTP source and a needed amount of chemical probe to be tested was added and the final assay volume was made up to 100 μl. Reaction was incubated at room temperature unless otherwise stated. Fluorescence units (FU) were measured using excitation and emission wavelengths of 465 nm and 535 nm, respectively. The same titer plate can be used several times to measure increases in fluorescence with time. The lipid transfer activity was presented as % lipid transfer/h/mg protein. To identify inhibitors, MTP activity was measured in the presence and absence of different compounds and percentage of inhibition was calculated.

MTP has been a target of therapeutic intervention for almost 20 years. Several pharmaceutical companies have heavily invested in identifying MTP antagonists to lower plasma cholesterol levels. However, therapeutic use of all currently developed compounds results in elevated plasma transaminases and hepatic lipid accumulation. As a consequence, MTP antagonists are only used for limited purposes, such as, lowering lipids in patients with familial hypercholesterolemia or controlling obesity in dogs. It is also currently being evaluated as a possible alternative to bariatric surgery to control blatant obesity, and liver transplantation to lower hyperlipidemias in familial hypercholesterolemia. These studies introduce a new concept for avoiding the side effects associated with MTP antagonists and advocates a novel method for identifying antagonists and antagonists used to treat hyperlipidemias and lead to new therapeutic modalities for the treatment of various hyperlipidemias and have immediate potential for translational use.

Inhibition of MTP is expected to decrease lipoprotein assembly and secretion thereby reducing plasma lipid levels. Use of specific antagonists that inhibit triglyceride transfer activity of MTP without affecting its phospholipid transfer activity may avoid toxicities associated with accumulation of lipids in different tissues. In addition, identification of such compounds would provide evidence for the existence of two functionally independent domains involved in phospholipid and triglyceride transfer in MTP.

The studies of the present invention demonstrate that there is one phospholipid site and another site for transfer of triglyceride. Identification of inhibitors that specifically inhibit either of these activities provide evidence that MTP contains two functionally independent domains involved in phospholipid and triglyceride transfer activities. Identification of small chemical compounds that inhibit triglyceride transfer activity of MTP without significantly affecting its phospholipid transfer activity might be ideal to reduce hyperlipidemia (high plasma lipids) and steatosis (high tissue lipids). The advantages of the method include ease, rapidity, sensitivity, avoidance of negatively charged lipids that inhibit MTP activity, versatility in studying different lipid transfer activities by purified and cellular MTP, ability to measure inhibitory activities of antagonists, and forestalling the use of radioactivity. For HTS purpose, the fluorescent assay described can be easily automated and used for large-scale through-put screening of small molecule libraries against MTP.

While the present invention has been described with reference to certain embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof.

What is claimed is:

1. A method for identifying an antagonist of microsomal triglyceride transfer protein (MTP), wherein the antagonist at least partially inhibits triglyceride transfer activity of MTP while not significantly inhibiting phospholipid transfer activity of MTP, said method comprising:
(a) identifying said antagonist in a primary high throughput (HTP) assay by:
(i) adding MTP into a cocktail comprising donor and acceptor lipid vesicles;
(ii) eliminating compounds which fluoresce and interfere with fluorescence detection by quenching or enhancing fluorescence;
(iii) identifying remaining compounds after step (ii) which at least partially inhibit triglyceride transfer activity of MTP; and
(iv) identifying remaining compounds after step (iii) which at least partially inhibit phospholipid transfer activity of MTP, resulting in identification of said antagonist;
(b) validating the antagonists identified in step (a)(iv) in a secondary radiolabeled lipid transfer assay by:
(i) preparing donor lipid vesicles comprising radiolabeled lipids and negatively charged cardiolipin;
(ii) preparing acceptor lipid vesicles;
(iii) incubating MTP with the donor lipid vesicles and acceptor lipid vesicles;
(iv) removing the donor lipid vesicles and MTP; and
(v) measuring net transfer of radiolabeled lipids by MTP from the donor to acceptor lipid vesicles by measuring radioactivity in the acceptor vesicles; and
(c) characterizing the biological activity of the identified antagonist obtained from the primary assay of step (a)(iv).

2. A method for treating hyperlipidemias or steatosis in a subject, said method comprising administering to the subject a therapeutically effective amount of an antagonist of MTP which at least partially inhibits triglyceride transfer activity of MTP while not significantly inhibiting phospholipid transfer activity of MTP.

3. An antagonist compound which at least partially inhibits triglyceride transfer activity of MTP while not significantly inhibiting phospholipid transfer activity of microsomal MTP.

4. A kit for measuring triglyceride and phospholipid transfer activity of MTP, said kit comprising a master mix and purified MTP, wherein the master mix comprises acceptor vesicles and fluorescence-labeled donor vesicles.
